(19) United States
(12) Patent Application Publication
(10) Pub. No.: US 2013/0269709 A1
(43) Pub. Date: Oct. 17, 2013

(54) WOUND DRESSING THAT CAN BE ADJUSTED TO THE WOUND SITE

(71) Applicant: Samuel L. Morris, III, Buffalo, NY (US)

(72) Inventor: Samuel L. Morris, III, Buffalo, NY (US)

(73) Assignee: MILSPORT MEDICAL, Buffalo, NY (US)

(21) Appl. No.: 13/576,993

(22) PCT Filed: Jan. 31, 2011
(86) PCT No.: PCT/US11/23084

§ 371 Date: Aug. 3, 2012

Related U.S. Application Data
(60) Provisional application No. 61/301,759, filed on Feb. 5, 2010.
(60) Provisional application No. 61/365,796, filed on Jul. 20, 2010.

(51) Int. Cl. A61F 15/00

(52) U.S. Cl. CPC A61F 15/008
USPC 128/845; 156/250

(57) ABSTRACT

The present invention relates to a wound dressing used for covering a wound site made of a backing layer, an adhesive layer, and a platform layer that together form a single unit; the backing layer is fastened to the platform layer by the adhesive layer and the platform layer is die cut into a pattern. The pieces of the pattern are selectively removable together with the adhesive layer, leaving the backing layer as part of the single unit, not selectively removed. The single unit would dressing serves to protect a wound site with no direct contact on the wound site.

The present invention related to the field of wound dressing

Wound dressings using an absorbent central area surround by a larger adhesive film are known. However, a need remains for a would dressing that can be more effectively applied to irregular wound sites, allowing for support, protection, and air circulation of the would site and body without direct contact with the wound site.

Therefore, a need remains for an improved would dressing configured for application

### SUMMARY OF THE INVENTION

In one embodiment the invention is a wound dressing used for covering a wound site. The wound dressing is comprised of layers which together form a single unit. The layers are a backing layer, an adhesive layer, and a platform layer. The backing layer is fastened to the platform layer by the adhesive layer. The platform layer is due cut into a pattern; and pieces of the pattern are selectively removable together with the adhesive layer leaving the backing layer as part of the single unit not selectively removed; so the single unit has not direct contact with the wound site. The single unit can be used in many different ways such as a cradle for holding an infant, an animal, or an isolated body part.

The wound site comprises of a body part that has sustained a trauma. The trauma to the body part comprises an ulcer, a bedsore, a blister, an incision, a burn, a fistula, a fracture, and a hematoma.

The backing layer of the single unit can be made of a variety of materials, for example, a fabric, a foam material, or a polymer material. The backing layer may have the property of vapor permeability it impermeability to liquids.

The platform layer can also be made of a variety of materials and one example could be a foam material. A desirable characteristic of the material in this layer is that of flexibility. Regardless of the composition of the material in the platform layer it must be die cut in at least two intersecting geometric shapes. Although, it is not necessary, a desirable form of the die cut is that the platform have at least two of the geometric shapes intersect. It is desired that the platform layer has a thickness of about 0.125 to about 5 inches.

The single unit can have a second adhesive applied to the platform layer on the side opposite from the backing layer. The second adhesive can be used to attach the single unit to a body part. The second adhesive can be protected by a removable cover. Another characteristic of the second adhesive is that it can be pressure sensitive.

Another embodiment of this invention is a method of applying a wound dressing to a wound site and surrounding body part, the method comprises: (a) providing a wound dressing comprising: (i) a. Backing layer, an adhesive layer, a platform layer, and a second adhesive layer that together form a single unit; said backing layer is attached to the platform layer by the adhesive layer, the second adhesive layer is attached to the platform layer, the platform layer is die cut into a pattern; (b) selectively removing pieces of the pattern together with the adhesive layer and leaving the backing layer as part of the single unit not selectively removed; (c) positioning the second adhesive layer over the would site and surrounding body part; and (d) applying a surface pressure to the single unit to conform the single unit to the body part surrounding the would site and; (e) secure the single unit it to the body part with the second adhesive.

In another embodiment this invention is a method of making a wound dressing comprising:
a. applying an adhesive to a backing layer;
b. attaching the backing layer in a step (a) to a platform layer;
c. optionally adding a two sided second adhesive to the platform layer in step (b); and
d. die cutting the platform layer and optional second adhesive layer into a patten to form a single unit would dressing.

Another embodiment of this invention is a method of covering a would site from an external pressure, the method comprising: (a) providing a wound dressing having (i) a. backing layer, an adhesive layer, and a platform layer that together form a single unit; said backing layer is attached to the platform layer by the adhesive layer, the platform layer is due cut into a pattern, (b) selectively removing pieces of the pattern together with the adhesive layer leaving the backing layer as part of the single unit not selectively removed; and (c) positioning a wound site in the area of the single unit where the platform was selectively removed leaving the body part cradled in the single unit wound dressing.

### BRIEF DESCRIPTION OF THE DRAWINGS

The invention will be further described with reference to the drawings, wherein corresponding reference characters indicate corresponding parts throughout several views, and wherein:

FIG. 1 is a side perspective view of a wound dressing configured and arranged in accordance with an implementation of the invention.

FIG. 2 is a top plan view of the wound dressing of FIG. 1.

FIG. 3 is a top plan view of the wound dressing in FIG. 1 with pieces of the pattern selectively removed.

FIG. 4 is a top plan view of the wound dressing in FIG. 1 with pieces of the pattern selectively removable.

FIG. 5 is a top plan view of an alternative embodiment of a wound dressing made in accordance with the invention.

FIG. 6 is view of the wound dressing FIG. 5 disassembled.

While the invention is amendable to various modifications and alternative forms, specifics thereof have been shown by way of example in the drawings and will be described in detail. It should be understood, however, that the intention is not limited to limit the invention to the particular embodiments described. On the contrary, the intention is to cover all modifications, equivalents, and alternatives falling within the spirit and scope of the invention.

### DETAILED DESCRIPTION

The present invention is directed to a would dressing used for covering a wound site comprising: a. backing layer, an adhesive layer, and a platform layer that together form a single unit and the platform layer can be selectively removed together with the adhesive layer leaving the backing layer as part of a single unit as a protective covering without direct contact with the wound site.

In some implementations the wound dressing comprises a backing layer, and an adhesive layer on the backing layer. The adhesive layer and backing layer form a perimeter around the platform layer and hold the platform layer in place on the body around the wound site without making contact with the wound site.

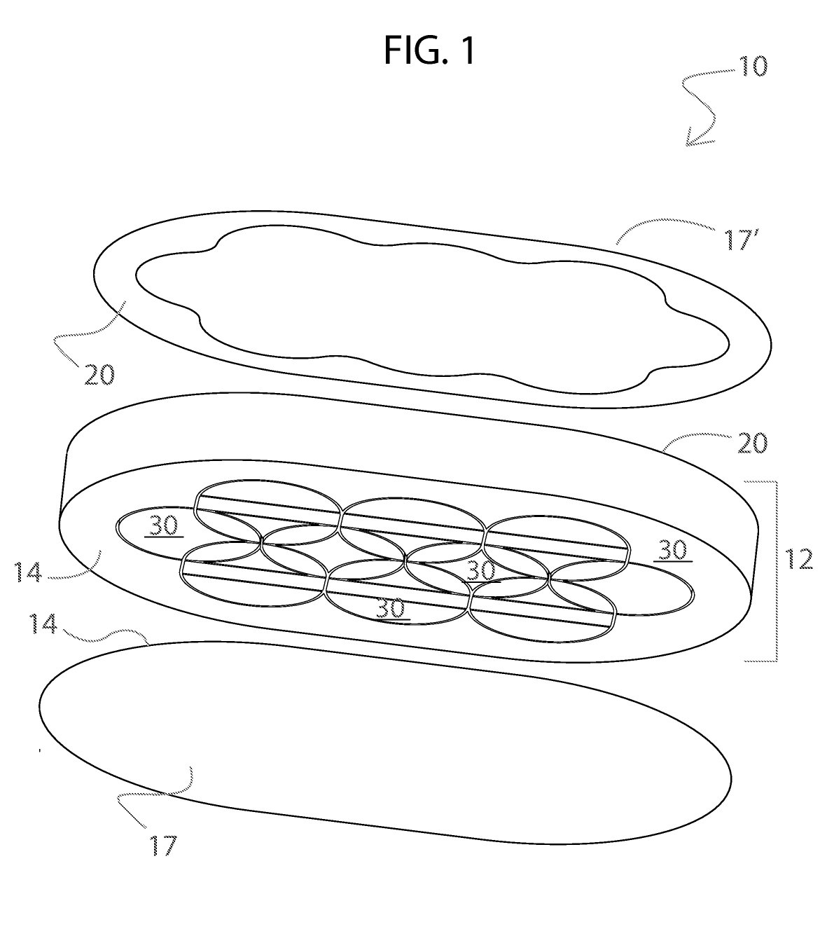

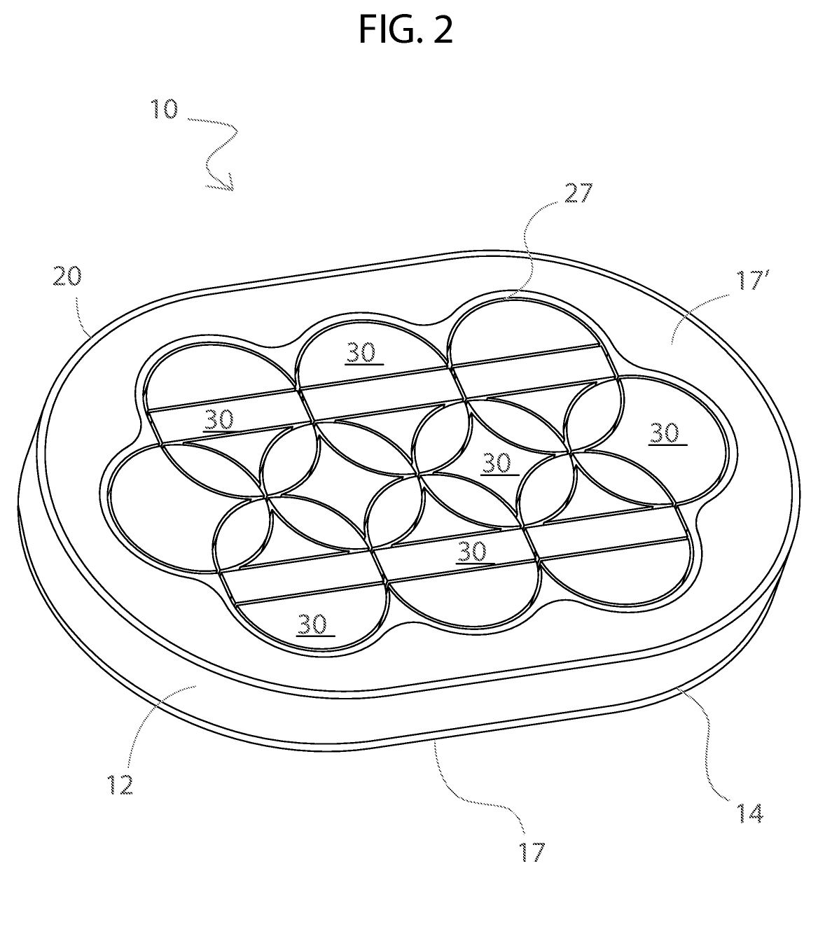

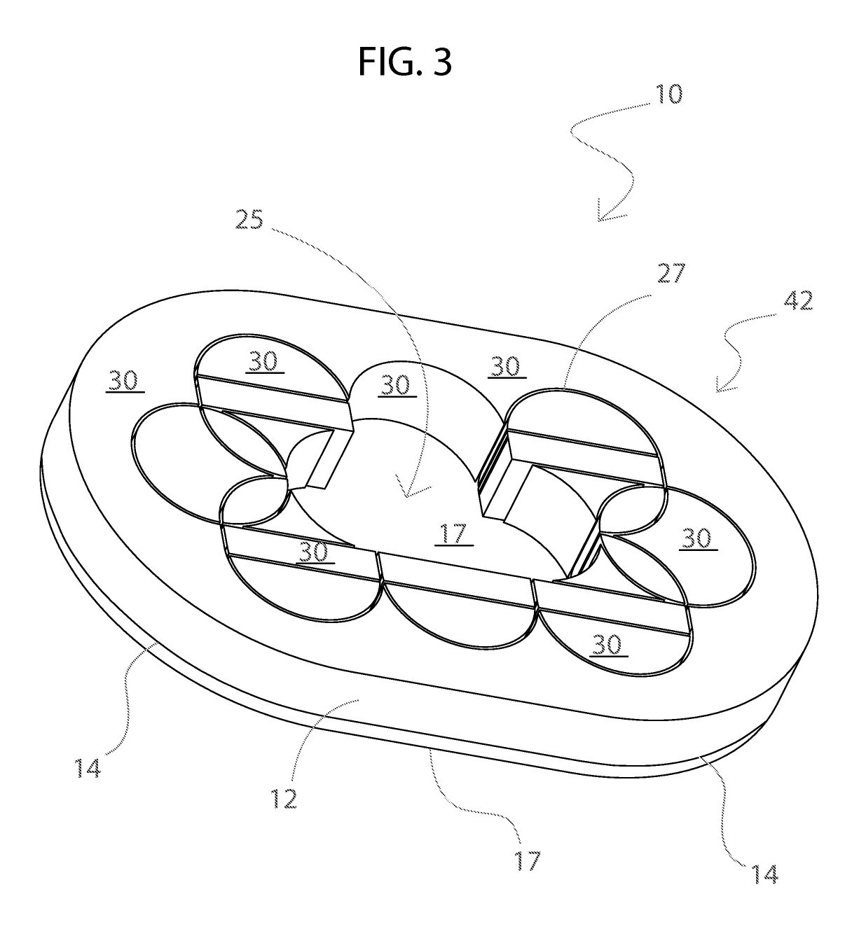

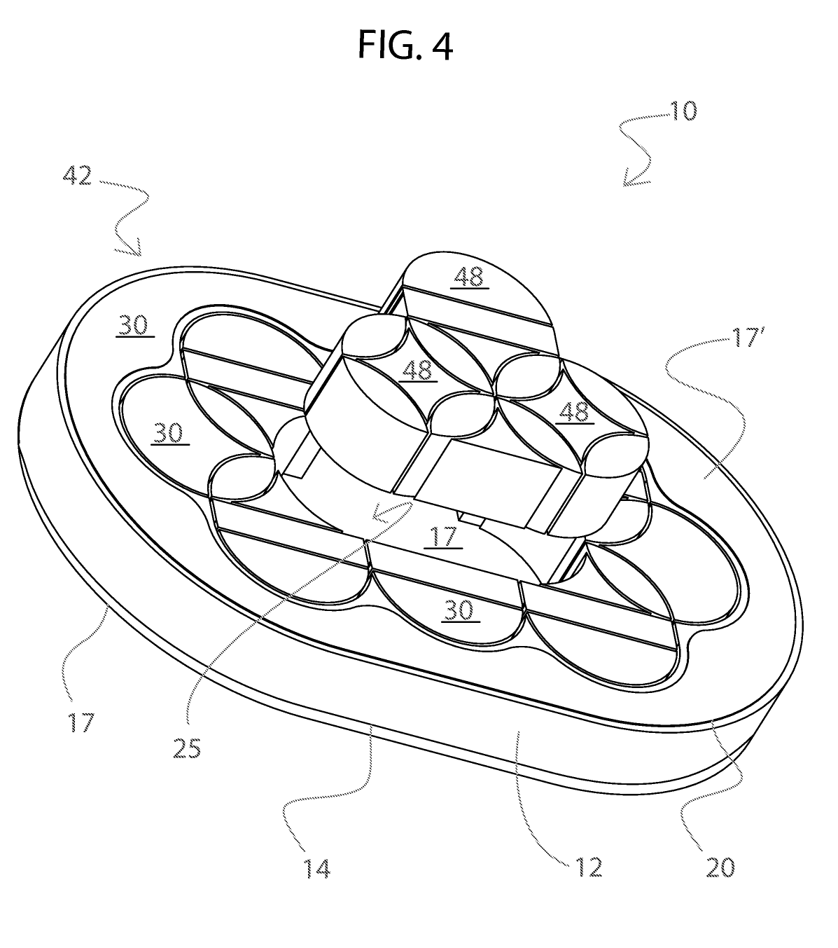

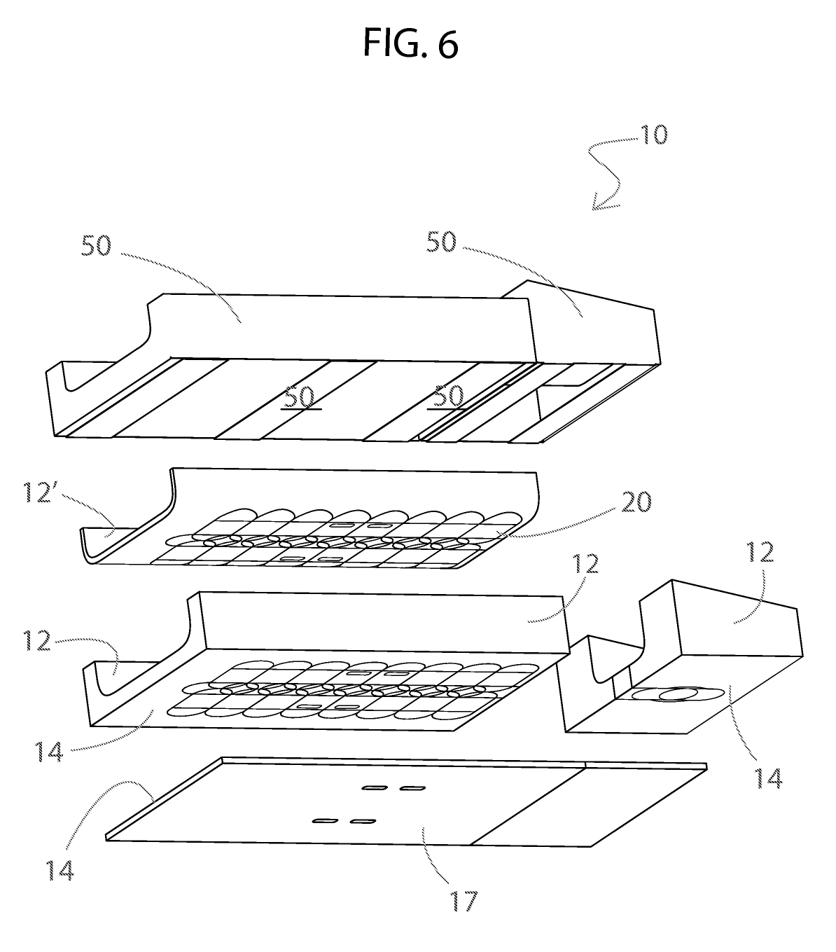

FIGS. 1-6 show examples of a wound dressing made in accordance with the invention. The wound dressing 10 includes a centrally located platform layer 12. The platform layer 12 is covered by a backing layer 17. Located between the backing layer and the platform layer is an adhesive layer 14. Optionally, located on the platform layer is a second adhesive layer 20.

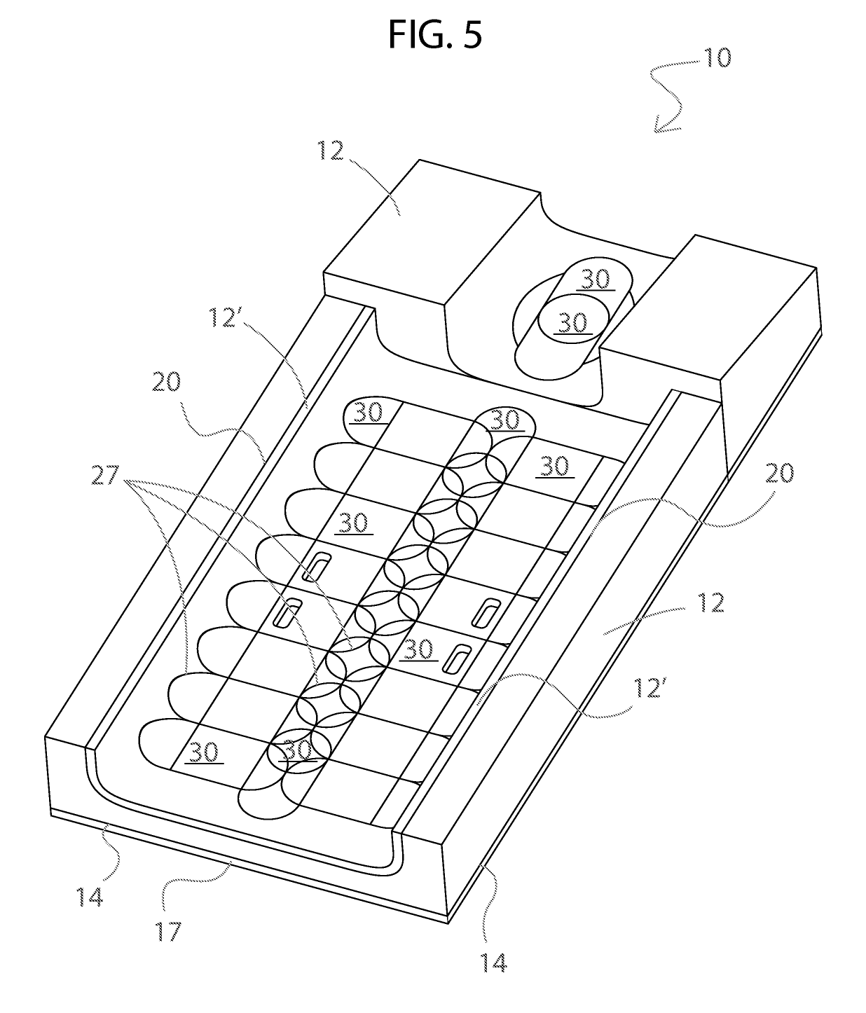

The backing layer can be made of a variety of materials. In one embodiment, the material has the characteristics of being flexible. In another embodiment (i.e. the embodiment is FIG. 5) the backing layer is a rigid. The backing layer can also have an absorbent characteristic. The backing layer may or may not be sterilized. The backing layer may be a non-woven anti-microbial stretch fabric. Suitable backing materials for the backing layer include, for example, non-woven fibrous webs, knits, silk, gauze, films, plastics, and other familiar materials. The backing materials may include polymeric elastic films. An example would include a lycra/PU film laminate.

The backing advantageously may transit moisture vapor at a rate equal to or greater than human skin.

In one embodiment, the backing layer is conformable to an anatomical surfaces. As such, when the backing layer is applied to an anatomical surface of a body, it conforms to the surface even when the surface is moved. The backing is also conformable to anatomical joints of a body. When the joint is flexed and then returned to its unflexed position, the backing layer can be made such that it stretches to accommodate the flexion of the joint, but is resilient enough to continue to conform to the joint when the joint is returned to its unflexed condition.

The backing layer can be made of multiple materials with different characteristics without diverging from the invention or deviating from the meaning of the term “backing” as used herein. Similarly, the backing layer can include multiple sub-layers, including films, webs, sheets, etc. Also, additional layers and films of other materials can be added between the materials described herein without deviating from the invention.

If the backing layer includes an absorbent material that material comprise a hydrocolloid and hydrogel pad and an additional transparent elastic polymeric film (e.g., urethane).

The platform layer has a series of geometric shapes that have been die cut into the platform layer, the platform layer is a single piece of material, such as, but not limited to, a polyurethane foam and the foam is an open cell medical grade. The material used to form the platform layer is generally substantially more rigid than the backing layer.

The geometric shapes require that they are cut in a way so as to be selectively removed together with the adhesive without disruption or tearing of the backing layer. The die cut geometric shapes provide a means for a controlled tear of the platform layer without the need for any tables to remove the platform layer during and/or after application of the dressing to a body.

In most implementations the platform layer geometric shapes are located in the center of the single unit. The configuration of the selectively removable geometric shapes allow the perimeter 30 of the wound dressing to be pressed around the body without contacting the wound site.

The geometric shapes can include, but are not limited to, circles, triangles, squares, or combinations thereof. In one embodiment the shapes are die cut to intersect.

The process of die cutting is computer-controlled and uses, for example but not limited to, manual hot wire, fast wire, and oscillating blade equipment for cutting the geometric shapes found in the platform layer. The geometric shapes may be screen printed prior to die cutting.

Selectively removable describes gripping and pulling (either manually or with the aide of a device such as, but not limited to, tweezers or pliers) precut sections of the platform layer and pulling said sections in a way that will expose the wound without limit to the size or shape of the wound. By selective removal of the foam the wound will be exposed to air circulation and can be easily treated with for example, surface antibiotics or cream as needed.

In one embodiment, the geometric patterns, such as those shown in FIG. 1-6, at least some of the geometric patterns narrow as they approach the perimeter of the wound dressing. A pattern is at least two intersecting geometric shapes.

The wound dressing requires an adhesive layer 14 between the backing layer and the platform layer. The adhesive layer can cover the surface of the platform layer or be a spot of adhesive on each individual die cut geometric shape. The adhesive is selectively removed together with the geometric patterns in the platform layer. The adhesive layer is generally a low adhesion coating. The adhesive is double sided and is characterized as adhering to the platform layer and the backing layer. The adhesive can be a hook and loop material. The adhesive can be selectively pulled away from the backing layer without destroying the backing layer while at the same time retaining adhesion to the platform layer. When the adhesive is selectively removed some of the adhesive may remain on the backing layer. Another characteristic is that the adhesive is a breathable PU film (polyurthethane).

The wound dressing is typically applied to a body by first cleaning the wound area and making use the area around the wound area is ready to receive a wound dressing. After determining the size and shape of the wound area the platform layer and adhesive layer are selectively removed.

Once the platform layer and adhesive layer of the wound dressing are selectively removed; only the backing layer provides significant rigidity to the wound dressing.

An optional second adhesive layer 20 may be applied to the platform layer on the side of the platform layer opposite the backing layer. The second adhesive layer 20 should be pressure sensitive to make it adhesive. The adhesive can be a hook and loop material. The pressure sensitive adhesive is usually reasonable skin compatible and “hypoallergenic”. Examples include, but are not limited to, iso-octyl acrylate: acrylamide copolymer, isooctyl acrylate: ethyleneoxide acrylate: acrylic acid terpolymer described in U.S. Pat. No. 4,737,410, the disclosure of which is hereby incorporated by reference is suitable. Additional useful adhesives are described in U.S. Pat. Nos. 4,310,509, and 4,313,557, the disclosures of which are hereby incorporated by reference. Inclusion of medicaments or antimicrobial agents in the adhesive is also contemplated, as described in U.S. Pat. Nos. 4,310,509 and 4,323,557, both of which are also hereby incorporated by reference.

The second adhesive layer can have a removable cover 50 that prevents the adhesive sticking prior to application to the body. The cover can be made of a paper like material or any material that has a property of being removable from the adhesive layer without disrupting the ability of the adhesive to adhere to another surface.

A wound is any body part that has experienced a trauma. The trauma can manifest as an ulcer, a bedsore, a blister, an incision, a burn, a fistula, a fracture, or a hematoma. As used herein a body refers to a living creature and a body part is a specific location the living creature.

As various changes could be made in the above constructions, compositions and methods without departing from the scope of the intervention as defined in the claims, it is intended that all matter contained in the above description or shown in the accompanying drawings be interpreted as illustrative and not a limiting sense.

1. A wound dressing used for covering a wound site comprising:
(a) a backing layer, an adhesive layer, and a platform layer that together forms a single unit;
said backing layer fastens to the platform layer by the adhesive layer;
the platform layer is cut into a pattern to define various sized and shaped wound sites; and
pieces of the pattern are selectively (a) removable together, or (b) removable and reattachable together, with at least a portion of the adhesive layer leaving the backing layer as part of the single unit not selectively removed to form a wound cavity formed single unit; and
said wound cavity formed single unit is positioned over the wound site, contacts skin surrounding the wound site and has no direct contact with the wound site.

2. The wound dressing of claim 1, wherein the wound site comprises a body part that has sustained a trauma.

3. The wound dressing of claim 2, wherein
the trauma to the body part comprises an ulcer, a bedsore, a blister, an incision, a burn, a fistula, a fracture, and a hematoma.

4. The wound dressing of claim 1, wherein the wound cavity formed single unit is a cradle for holding an infant.

5. The wound dressing of claim 1, wherein the wound cavity formed single unit is a cradle for holding an isolated body part.

6. The wound dressing of claim 1, wherein the backing layer comprises a fabric, a foam material, a polymer material.

7. The wound dressing of claim 1, wherein the backing layer is vapor permeable but impermeable to liquids.

8. The wound dressing of claim 1, wherein the platform layer comprises a foam material.

9. The wound dressing of claim 1, wherein the platform layer is flexible.

10. The wound dressing of claim 1, wherein the platform layer is die cut in at least two intersecting geometric shapes.

11. The wound dressing of claim 10, wherein the geometric shapes are screen printed on the platform layer's side opposite from the backing layer.

12. The wound dressing of claim 1, wherein the platform layer has a thickness of about 0.125 to about 5 inches.

13. The wound dressing of claim 1, wherein a second adhesive is applied to the platform layer on the side opposite from the backing layer.

14. The wound dressing of claim 13, wherein the second adhesive is used to attach the wound cavity formed single unit to the body part.

15. The wound dressing of claim 14, wherein the second adhesive is protected by a removable cover.

16. The wound dressing of claim 13, wherein the second adhesive is pressure sensitive.

17. The wound dressing of claim 13, wherein the adhesive is a breathable PU film.

18. A method of applying a wound dressing to a wound site and surrounding body part, the method comprising:
(a) providing a wound dressing comprising:
a backing layer, an adhesive layer, a platform layer, and a second adhesive layer that together form a single unit;
said backing layer attaches to the platform layer by the adhesive layer,
the second adhesive layer is attached to the platform layer's side opposite from the backing layer,
the platform layer is cut into a pattern to define various sized and shaped wound sites;
(b) selectively removing or temporally removing pieces of the pattern together with at least a portion of the adhesive layer and leaving the backing layer as part of the single unit not selectively removed to form a wound cavity formed single unit;
(c) positioning the second adhesive layer over the body part surrounding the wound site; and
(d) applying a surface pressure to the wound cavity formed single unit to conform the wound cavity formed single unit to the body part surrounding the wound site; and
(e) secure the wound cavity formed single unit to the body part with the second adhesive.

19. A method of making a wound dressing comprising:
(a) applying an adhesive to a backing layer;
(b) attaching the backing layer in step (a) to a platform layer;
(c) optionally adding a two sided second adhesive to the platform layer side opposite from the backing layer in step (b);
(d) cutting the platform layer and optional second adhesive layer into a pattern to form a single unit wound dressing.

20. A method of covering a wound site from an external pressure, the method comprising:
(a) providing a wound dressing having a backing layer, an adhesive layer, and a platform layer that together form a single unit;
said backing layer attaches to the platform layer by the adhesive layer,
the platform layer is cut into a pattern to define various sized and shaped wound sites,
(b) selectively removing or temporally removing pieces of the pattern together with at least a portion of the adhesive layer leaving the backing layer as part of the single unit not selectively removed to form a wound cavity formed single unit; and
(c) positioning a wound site in the area of the operable single unit where the platform was selectively removed leaving the body part cradled in the wound cavity formed single unit wound dressing.

21. (canceled)

22. (canceled)

23. The wound dressing of claim 1 wherein the cut pattern is formed through a die cut.
